(19) United States
(12) Patent Application Publication
(10) Pub. No.: US 2019/0013097 A1
(43) Pub. Date: Jan. 10, 2019

(54) METHOD, SYSTEM, AND NON-TRANSITORY COMPUTER-READABLE RECORDING MEDIUM FOR PROVIDING MEDICAL SERVICE

(71) Applicant: Hyung Tay RHO, Seoul (KR)

(72) Inventor: Hyung Tay RHO, Seoul (KR)

(21) Appl. No.: 16/125,833

(22) Filed: Sep. 10, 2018

Related U.S. Application Data
(63) Continuation of application No. PCT/KR2017/002637, filed on Mar. 10, 2017.

(30) Foreign Application Priority Data
Mar. 10, 2016 (KR) 10-2016-0029067

(51) Int. Cl. G16H 40/20; G06F 19/00; G16H 10/60; G16H 40/63; H04W 4/029

(52) U.S. Cl. CPC G16H 40/20; G06F 19/709; H04W 4/029; G16H 40/63; G16H 10/60

(57) ABSTRACT

The present invention relates to a method, system, and non-transitory computer-readable recording medium for providing a medical service. According to one aspect of the invention, there is provided a method for providing a medical service, comprising the steps of: determining whether a patient carrying a patient device enters a hospital, with reference to information on a location of the patient device; acquiring information on a medical appointment or an electronic medical record (EMR) of the patient, when it is determined that the patient enters the hospital; and establishing a medical resource allocation policy personalized for the patient, with reference to the acquired information on the medical appointment or electronic medical record.

### CROSS-REFERENCE TO RELATED APPLICATION(S)

This application is a continuation application of Patent Cooperation Treaty (PCT) International Application Serial No. PCT/KR2017/002637, filed on Mar. 10, 2017, which claims priority to Korean Patent Application Serial No. 10-2016-0029067, filed on Mar. 10, 2016. The entire contents of PCT International Application Serial No. PCT/KR2017/002637 and Korean Patent Application Serial No. 10-2016-0029067 are hereby incorporated by reference.

### FIELD OF THE INVENTION

The present invention relates to a method, system, and non-transitory computer-readable recording medium for providing a medical service.

### BACKGROUND

Conventionally, medical resources of a hospital or a clinic have been managed commonly on the basis of intentional manipulation according to subjective judgment of a medical attendant or an assistant medical attendant, or on the basis of an active request of a patient. For example, a medical attendant or a bed can be allocated to a patient visiting a hospital only after the patient has completed a reception procedure, and medicines or medical equipment can be allocated to the patient only after the medical attendant reads information on a medical appointment or an electronic medical record of the patient and recognizes the contents thereof to make some manipulation or give some instruction.

However, according to the above conventional technology, there is a limitation that medical resources cannot be properly and efficiently allocated in a small clinic with deficient medical resources, or in a large hospital visited by a large number of patients, and there is also a problem that patients may be dissatisfied with long waiting time due to delayed progression of medical care.

In order to solve the above problems, a technique has been introduced in which, when a patient completes a reception procedure or the like through a counter or kiosk, notification information thereon can be automatically transmitted to a medical attendant. However, even with the above technique, processes for allocating medical resources cannot but progress slowly because an action or request of the patient or judgment or instruction of the medical attendant is still required to allocate the medical resources, and thus there is a limitation in increasing the efficiency of utilizing the medical resources.

Meanwhile, conventionally, detailed (or friendly) guidance information has not been provided commonly to a patient who visits a hospital to receive medical care. Even when such guidance information is provided, only hospital-friendly guidance information (e.g., information described with difficult terminology for the patient to understand) has been limitedly provided. This has caused various side effects (e.g., increased patient complaints) due to medical information asymmetry between the hospital and the patient. Further, even when a hospital intends to provide patient-friendly guidance information in detail, there is a problem that the efficiency of the hospital work is lowered because the time and effort therefor are excessively required.

In this regard, the present inventor(s) suggest a technique that may efficiently allocate medical resources using information on a location of a patient and information on a medical appointment or an electronic medical record of the patient, and may provide the patient with guidance information suitable for conditions and contexts of medical care.

### SUMMARY OF THE INVENTION

One object of the present invention is to solve all the above-described problems in the prior art.

Another object of the invention is to efficiently allocate medical resources using information on a location of a patient and information on a medical appointment or an electronic medical record of the patient, and to provide the patient with guidance information suitable for conditions and contexts of medical care, by determining whether a patient carrying a patient device enters a hospital, with reference to information on a location of the patient device; acquiring information on a medical appointment or an electronic medical record (EMR) of the patient, when it is determined that the patient enters the hospital; and establishing a medical resource allocation policy personalized for the patient, with reference to the acquired information on the medical appointment or electronic medical record.

The representative configurations of the invention to achieve the above objects are described below.

According to one aspect of the invention, there is provided a method for providing a medical service, comprising the steps of: determining whether a patient carrying a patient device enters a hospital, with reference to information on a location of the patient device; acquiring information on a medical appointment or an electronic medical record (EMR) of the patient, when it is determined that the patient enters the hospital; and establishing a medical resource allocation policy personalized for the patient, with reference to the acquired information on the medical appointment or electronic medical record.

According to another aspect of the invention, there is provided a system for providing a medical service, comprising: a location recognition unit configured to determine whether a patient carrying a patient device enters a hospital, with reference to information on a location of the patient device; an information management unit configured to acquire information on a medical appointment or an electronic medical record (EMR) of the patient, when it is determined that the patient enters the hospital; and a medical resource management unit configured to establish a medical resource allocation policy personalized for the patient, with reference to the acquired information on the medical appointment or electronic medical record.

In addition, there are further provided other methods and systems to implement the invention, as well as non-transitory computer-readable recording media having stored thereon computer programs for executing the methods.

According to the invention, it is possible to efficiently allocate medical resources using information on a location of a patient and information on a medical appointment or an electronic medical record of the patient, thereby preventing the medical resources from being wasted and reducing the patient's time for medical care or waiting.

According to the invention, it is possible to accurately and promptly allocate medical resources without an action or request of a patient or judgment or manipulation of a medical attendant.

According to the invention, it is possible to automatically provide a patient with guidance information suitable for conditions and contexts of the patient's medical care in a patient-friendly form, without needing a medical attendant or an assistant medical attendant to take the time and effort to provide the patient with the guidance information, thereby minimizing various side effects that may be caused by medical information asymmetry between a hospital and the patient, and increasing the patient's satisfaction and confidence in medical services.

### BRIEF DESCRIPTION OF THE DRAWINGS

FIG. 1 schematically shows the configuration of an entire system for providing a medical service according to one embodiment of the invention.

FIG. 2 specifically shows the internal configuration of a main system according to one embodiment of the invention.

FIG. 3 conceptually shows how to determine whether a patient enters a hospital according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the locations or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

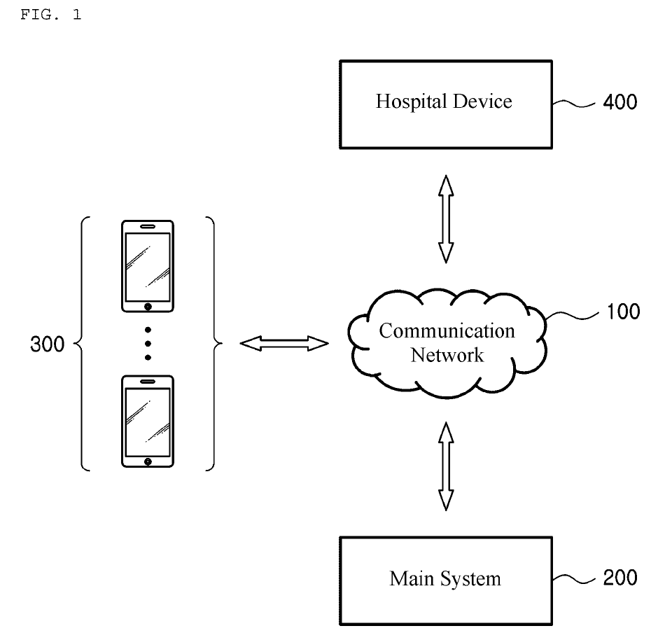

Configuration of the entire system FIG. 1 schematically shows the configuration of an entire system for providing a medical service according to one embodiment of the invention.

As shown in FIG. 1, the entire system according to one embodiment of the invention may comprise a communication network 100, a main system 200, a patient device 300, and a hospital device (or hospital server) 400.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, Long Term Evolution (LTE) communication, Bluetooth communication (more specifically, Bluetooth Low Energy (BLE) communication), infrared communication, and ultrasonic communication.

Next, according to one embodiment of the invention, the main system 200 may interwork with the patient device 300 or the hospital device (or hospital server) 400 on the communication network 100, and may function to efficiently allocate medical resources using information on a location of a patient and information on a medical appointment or an electronic medical record of the patient, by determining whether a patient carrying the patient device 300 enters a hospital, with reference to information on a location of the patient device 300; acquiring information on a medical appointment or an electronic medical record (EMR) of the patient, when it is determined that the patient enters the hospital; and establishing a medical resource allocation policy personalized for the patient, with reference to the acquired information on the medical appointment or electronic medical record.

The configuration and function of the main system 200 according to the invention will be discussed in more detail below.

Next, according to one embodiment of the invention, the patient device 300 is digital equipment that may function to connect to and then communicate with the main system 200 or the hospital device (or hospital server) 400, and any type of digital equipment having a memory means and a microprocessor for computing capabilities, such as a smart phone and a tablet PC, may be adopted as the patient device 300 according to the invention.

Specifically, according to one embodiment of the invention, the patient device 300 may include a communication module (not shown) for wireless communication with a wireless communication module (e.g., a BLE beacon) installed in a hospital, a sensor module (not shown) that may sense the strength of a radio signal received from the wireless communication module installed in the hospital (e.g., a RSSI (Received Signal Strength Indication) sensor module), a location recognition module (not shown) that may acquire information on a location of the patient device 300 (e.g., a GPS module, an IMU (Inertial Measurement Unit), etc.), and the like.

For example, a wireless communication module (not shown) that may be included in the patient device 300 according to one embodiment of the invention may be assumed to be a wireless communication module with BLE technology, a WLAN (Wireless LAN) communication module, an ultrasonic communication module, an ACR (Access Control Router) module, or the like. However, it is noted that the configuration of the patient device 300 according the invention is not necessarily limited thereto, and may be changed without limitation as long as the objects of the invention may be achieved.

Meanwhile, according to one embodiment of the invention, the patient device 300 may include an application to assist a patient to receive medical services according to the invention. The application may be downloaded from the main system 200 or an external application distribution server (not shown).

Next, according to one embodiment of the invention, the hospital device (or hospital server) 400 is digital equipment that may function to connect to and then communicate with the main system 200 or the patient device 300, and may function to manage medical resources in a hospital according to a medical resource allocation policy established and provided (or distributed) by the main system 200.

Configuration of the Main System

Hereinafter, the internal configuration of the main system 200 according to the invention and the functions of the respective components thereof will be discussed.

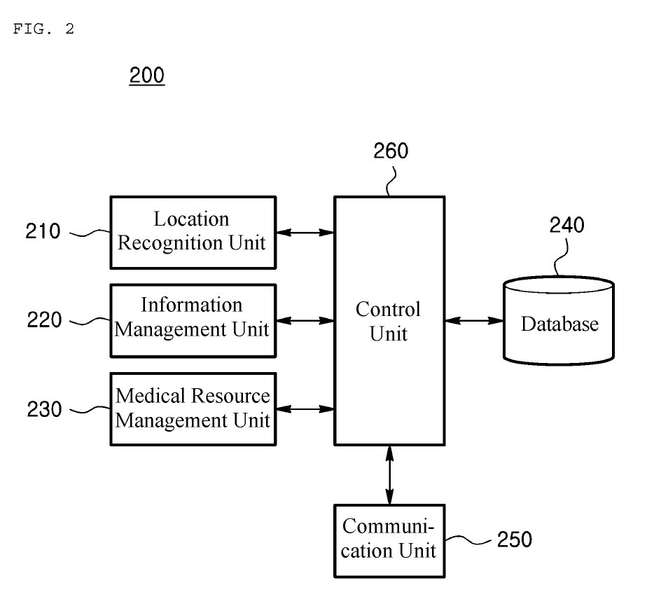

FIG. 2 specifically shows the internal configuration of the main system 200 according to one embodiment of the invention.

As shown in FIG. 2, the main system 200 according to one embodiment of the invention may comprise a location recognition unit 210, an information management unit 220, a medical resource management unit 230, a database 240, a communication unit 250, and a control unit 260. According to one embodiment of the invention, at least some of the location recognition unit 210, the information management unit 220, the medical resource management unit 230, the database 240, the communication unit 250, and the control unit 260 may be program modules to communicate with the patient device 300 or the hospital device (or hospital server) 400. The program modules may be included in the main system 200 in the form of operating systems, application program modules, and other program modules, while they may be physically stored in a variety of commonly known storage devices. Further, the program modules may also be stored in a remote storage device that may communicate with the main system 200. Meanwhile, such program modules may include, but not limited to, routines, subroutines, programs, objects, components, data structures, and the like for performing specific tasks or executing specific abstract data types as will be described below in accordance with the invention.

First, according to one embodiment of the invention, the location recognition unit 210 may function to determine whether a patient carrying the patient device 300 enters a hospital, with reference to information on a location of the patient device 300.

Specifically, the location recognition unit 210 according to one embodiment of the invention may acquire the information on the location of the patient device 300 from a wireless communication module (e.g., a BLE beacon) in the hospital, which performs short-range wireless communication with the patient device 300. Further, the location recognition unit 210 according to one embodiment of the invention may recognize the location of the patient device 300 on the basis of information on a magnetic field measured by an inertial measurement unit (IMU) provided in the patient device 300. However, the location recognition techniques applicable to the invention are not necessarily limited thereto, and may be changed without limitation as long as the objects of the invention may be achieved.

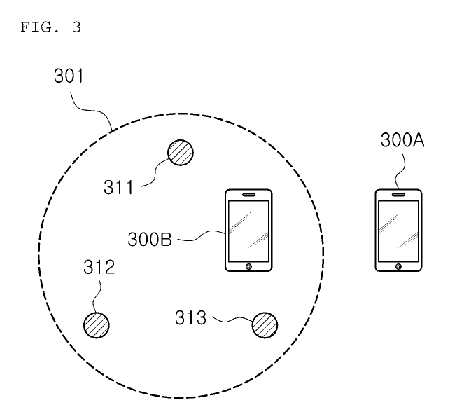

FIG. 3 conceptually shows how to determine whether a patient enters a hospital according to one embodiment of the invention.

Referring to FIG. 3, when a patient enters a hospital 301, information on a patient device carried by the patient may be acquired as the patient device communicates with at least one wireless communication module 311, 312, 313 installed in the hospital, and it may be determined whether the patient enters the hospital 301 on the basis of the acquired information.

For example, in the embodiment of FIG. 3, a patient device 300A may be located far from the wireless communication module 311, 312, 313 installed in the hospital 301 before the patient enters the hospital 301, and the strength of a radio signal transmitted and received between the patient device 300A and the wireless communication module 311, 312, 313 may be accordingly small. On the other hand, a patient device 300B may be located near the wireless communication module 311, 312, 313 installed in the hospital 301 after the patient enters the hospital 301, and the strength of a radio signal transmitted and received between the patient device 300B and the wireless communication module 311, 312, 313 may be accordingly large. Thus, the location recognition unit 210 according to one embodiment of the invention may determine whether the patient enters the hospital 301 on the basis of the strength (more specifically, RSSI) of the radio signal recognized as above.

Next, according to one embodiment of the invention, the information management unit 220 may function to acquire information on a medical appointment or an electronic medical record (EMR) of the patient, when it is determined that the patient enters the hospital.

Further, the information management unit 220 according to one embodiment of the invention may function to transmit at least a part of the information on the medical appointment or electronic medical record of the patient to the patient device 300 of the corresponding patient, so that the patient may be provided with information related to medical services to be provided to the patient. For example, the information that may be transmitted to the patient device 300 may include a date and time of the appointment, a medical attendant in charge, current medical care conditions, future medical care plans, precautions, billing information, and the like.

Next, according to one embodiment of the invention, the medical resource management unit 230 may function to establish a medical resource allocation policy personalized for the patient who enters the hospital, with reference to the acquired information on the medical appointment or electronic medical record. Here, according to one embodiment of the invention, the medical resources may include medical attendants, medicines, medical equipment, beds, medical care hours, and the like. However, it is noted that the types of medical resources manageable in the invention are not necessarily limited thereto, and may be changed without limitation as long as the objects of the invention may be achieved.

Further, according to one embodiment of the invention, the medical resource management unit 230 may establish a medical resource allocation policy personalized for the patient who enters the hospital, with further reference to information on a current location of the patient or a past location history of the patient.

Meanwhile, the medical resource management unit 230 according to one embodiment of the invention may function to distribute the established medical resource allocation policy to at least one hospital device (or hospital server) 400 that manages the medical resources of the corresponding hospital.

For example, it may be assumed that a patient A visits a hospital to receive dental care. In this case, when it is determined that the patient A enters the hospital, the medical resource management unit 230 according to one embodiment of the invention may refer to medical appointment information indicating that the patient A has made an appointment to receive the dental care from a doctor A, and EMR information indicating that the patient A has recently been undergoing orthodontic treatment, and may establish a medical resource allocation policy to secure a chair for the patient A and medical instruments for orthodontic treatment in advance, and to allocate the doctor A to the patient A. The hospital device (or hospital server) 400 may receive the established medical resource allocation policy and then transmit a message to an assistant medical attendant to advise him/her to prepare the chair for the patient A and the medical instruments for orthodontic treatment, and a message to the doctor A to remind him/her of the medical appointment with the patient A or inform that the patient A has arrived at the hospital.

As another example, it may be assumed that a patient B visits a hospital to a health checkup. In this case, when it is determined that the patient B enters the hospital, the medical resource management unit 230 according to one embodiment of the invention may refer to medical appointment information indicating that the patient B has made an appointment to receive an MRI scan, and may establish a medical resource allocation policy to secure medical equipment for MRI scanning for the patient B in advance. The hospital device (or hospital server) 400 may receive the established medical resource allocation policy and then add a time slot for the patient B to a schedule for use of the medical equipment for MRI scanning.

As yet another example, it may be assumed that a patient C visits a hospital without any medical appointment. In this case, the medical resource management unit 230 according to one embodiment of the invention may refer to EMR information indicating that the patient C has frequently received internal medical care in the past, and location information indicating that the patient C is showing a movement pattern similar to those shown when he/she received internal medical care in the past, and may establish a medical resource allocation policy to secure medical resources for internal medicine for the patient C in advance. The hospital device (or hospital server) 400 may receive the established medical resource allocation policy and then add a time slot for the patient C to a schedule for internal medical care.

Meanwhile, according to one embodiment of the invention, the main system 200 may function to provide a patient with guidance information suitable for conditions and contexts of medical care.

Specifically, according to one embodiment of the invention, the information management unit 220 may function to generate guidance information to be provided to a patient, with reference to information on a medical appointment or an electronic medical record of the patient and context information related to medical care of the patient, and to determine a time or place in which the generated guidance information is to be provided to the patient. Here, at least a part of the generated guidance information may be matched to at least one of the information on the medical appointment or electronic medical record of the patient and the context information of the patient, among information stored in the database 240, and extracted from the database 240.

Further, according to one embodiment of the invention, the information management unit 220 may function to provide the generated guidance information to the device of the patient, when the determined time has arrived or the patient is located at the determined place. For example, the information management unit 220 according to one embodiment of the invention may determine whether the patient enters the hospital, with reference to information on a location of the device 300 of the patient, and may provide guidance information generated for the patient to the device 300 of the patient, when it is determined that the patient enters the hospital.

Here, according to one embodiment of the invention, the context information may include a medical history of the patient at the hospital or other hospitals, a disease of the patient, a medicine taken by the patient, a lifestyle of the patient, a biometric signal acquired from the patient, and the like. However, it is noted that the context information according the invention is not necessarily limited thereto, and may be changed without limitation as long as the objects of the invention may be achieved.

Further, according to one embodiment of the invention, the guidance information provided to the patient refers to information considered to be useful to the patient in relation to the patient's medical care conditions, and may comprise various types of objects such as text, images, video, and audio composed in patient-friendly forms. Specifically, according to one embodiment of the invention, at least some of the objects constituting the guidance information stored in the database 240 may have a correlative relationship or matching relationship with a specific object that may be included in the information on the medical appointment or electronic medical record or the context information, and the correlative relationship or matching relationship may be referred to when the guidance information to be provided to the patient is extracted from the database 240. According to one embodiment of the invention, the guidance information stored in the database 240 may be extracted or processed from the information on the electronic medical record, and may be extracted or processed from a variety of information that may be acquired from an external server (not shown) via the communication network 100.

Hereinafter, the embodiments in which a patient is provided with guidance information suitable for conditions and contexts of medical care will be discussed in detail.

For example, it may be assumed that a patient D visits a hospital to receive cavitation treatment. In this case, according to one embodiment of the invention, the patient D may be provided with guidance information related to information on a medical appointment or an electronic medical record of the patient D or context information of the patient D. Specifically, according to one embodiment of the invention, patient-friendly guidance information such as “Details of resin filling treatment,” which is matched to hospital-friendly memorandum information such as “e-max inlay” or “R.F. (premolar)” written in the (electronic) medical record by a medical attendant, may be extracted from the database 240 and provided to the device 300 of the patient D. According to one embodiment of the invention, a variety of guidance information, such as a treatment progress rate, treatment plans, completed treatment, remaining treatment, the expected number of visits for future treatment, actual treatment photographs, a doctor's opinion on treatment, precautions before treatment, precautions after treatment, and materials used in treatment, may be further provided to the patient D.

As another example, it may be assumed that a patient E visits a hospital to receive neural tube disinfection. In this case, according to one embodiment of the invention, it may be determined whether the patient E enters the hospital or where the patient E is located in the hospital, on the basis of location information and identification information acquired from the device 300 carried by the patient E. Further, according to one embodiment of the invention, when it is determined that the patient E has just entered the hospital and is located at a desk or waiting room or seated on a chair, guidance information on neural tube disinfection to be shortly performed may be provided to the device 300 of the patient E. When the patient E is away from the chair after the neural tube disinfection has been completed and is again located at the desk or waiting room, guidance information on symptoms that may be present following the just completed neural tube disinfection, or guidance information on future treatment plans, may be provided to the device 300 of the patient E.

As yet another example, it may be assumed that a patient F visits a hospital to receive a checkup for implant surgery, and a surgical plan is established to perform maxillary sinus lift and implant placement through a crestal approach after 7 days as a result of the checkup. In this case, according to one embodiment of the invention, guidance information on precautions or tips that a hypertensive patient should know in relation to implant surgery may be provided to the patient F, on the basis of context information that aspirin for hypertension treatment is orally administered to the patient F. Specifically, according to one embodiment of the invention, before the implant surgery is performed, guidance information such as “Aspirin may disturb hemostasis, so you should stop taking aspirin.” or “You have stopped taking aspirin, so you should strictly obey lifestyle guidelines for hypertensives.” may be provided to the device 300 of the patient F in the form of a notification, from when one week remains until the scheduled implant surgery. Further, according to one embodiment of the invention, on the day when the implant surgery is performed, guidance information on anticipated hemostatic disorder symptoms, timing for resumption of oral aspirin administration, lifestyle guidelines, diets, and the like may be provided to the device 300 of the patient F. Furthermore, according to one embodiment of the invention, on the day when treatment after the implant surgery is performed, the contents of guidance information to be provided to the patient F may be dynamically determined on the basis of a recovery state described in the electronic medical record of the patient F (e.g., whether the recovery is normal or whether there is a nosebleed due to the maxillary sinus lift). In addition, according to one embodiment of the invention, on the day when surgical thread removal after the implant surgery is performed, guidance information such as “Bleeding due to thread removal may not stop in some cases, so please be informed of the following tips.” may be provided to the device 300 of the patient F on the basis of context information that the patient F has resumed oral administration of aspirin.

As still another example, it may be assumed that a patient G receives the same implant surgery as the patient F. In this case, according to one embodiment of the invention, on the basis of context information that the patient G is suffering from diabetes and undergoing diabetes control treatment with the help of another hospital, and that his/her postprandial blood glucose level is 150 mg/dL, guidance information related to keywords such as “diabetes”, “under control”, and “postprandial 150”, among guidance information stored in the database 240 and related to implant surgery, may be determined to be provided to the patient G. Specifically, according to one embodiment of the invention, pre-surgery guidance information such as “Diabetic patients are very susceptible to infection, and those having a blood glucose level of 150 mg/dL belong to a risk group, so gargling with a hexamedine gargle solution (or a solution for bacterial reduction) is necessary before the implant surgery.” may be provided to the device 300 of the patient G, and guidance information on precautions, diets, lifestyle guidelines, and the like after the implant surgery may be further provided. Meanwhile, according to one embodiment of the invention, the contents of guidance information provided to the patient G may be dynamically determined on the basis of a result of monitoring (i.e., measuring) blood glucose levels of the patient G, which is acquired in real time or periodically from the device 300 of the patient G or a server (not shown) of another hospital.

Meanwhile, according to one embodiment of the invention, the database 240 may store information on a location of the patient device 300, information on a medical appointment of the patient, information on an electronic medical record of the patient, information on a past location history of the patient, and the like. Further, according to one embodiment of the invention, the database 240 may store guidance information that may be provided to the patient, and information on a correlative relationship or matching relationship between the guidance information and the information on the medical appointment or electronic medical record or the context information. Although FIG. 2 shows that the database 240 is incorporated in the main system 200, the database 240 may be configured separately from the main system 200 as needed by those skilled in the art to implement the invention. Meanwhile, the database 240 according to the invention encompasses a computer-readable recording medium, and may refer not only to a database in a narrow sense but also to a database in a broad sense including file system-based data records and the like. The database 240 according to the invention may be even a collection of simple logs if one can search and retrieve data from the collection.

Next, the communication unit 250 according to one embodiment of the invention may function to enable data transmission/reception from/to the main system 200.

Lastly, the control unit 260 according to one embodiment of the invention may function to control data flow among the location recognition unit 210, the information management unit 220, the medical resource management unit 230, the database 240, and the communication unit 250. That is, the control unit 260 according to the invention may control data flow into/out of the main system 200 or data flow among the respective components of the main system 200, such that the location recognition unit 210, the information management unit 220, the medical resource management unit 230, the database 240, and the communication unit 250 may carry out their particular functions, respectively.

The embodiments according to the invention as described above may be implemented in the form of program instructions that can be executed by various computer components, and may be stored on a non-transitory computer-readable recording medium. The computer-readable recording medium may include program instructions, data files, data structures and the like, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be changed to one or more software modules to perform the processes of the present invention, and vice versa.

Although the present invention has been described in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for providing a medical service, comprising the steps of:
determining whether a patient carrying a patient device enters a hospital, with reference to information on a location of the patient device;
acquiring information on a medical appointment or an electronic medical record (EMR) of the patient, when it is determined that the patient enters the hospital;
establishing a medical resource allocation policy personalized for the patient, with reference to the acquired information on the medical appointment or electronic medical record;
generating guidance information to be provided to the patient, with reference to the information on the medical appointment or electronic medical record of the patient and context information of the patient, and determining a time or place in which the generated guidance information is to be provided to the patient; and
providing the generated guidance information to the device of the patient, when the determined time has arrived or the patient is located at the determined place,
wherein the context information includes information on at least one of a medical history of the patient at the hospital or other hospitals, a disease of the patient, a medicine taken by the patient, a lifestyle of the patient, and a biometric signal acquired from the patient.

2. The method of claim 1, wherein the location of the patient device is determined on the basis of information acquired from the patient device or at least one wireless communication module that is installed in the hospital and performs short-range wireless communication with the patient device.

3. The method of claim 1, wherein the location of the patient device is determined on the basis of information acquired from an inertial measurement unit (IMU) provided in the patient device.

4. The method of claim 1, wherein the medical resources include at least one of medical attendants, medicines, medical equipment, beds, and medical care hours.

5. The method of claim 1, wherein in the establishing step, the medical resource allocation policy personalized for the patient is established with further reference to information on at least one of a current location of the patient and a past location history of the patient.

6. The method of claim 1, further comprising the step of:
distributing the established medical resource allocation policy to at least one hospital device or hospital server that manages the medical resources of the hospital.

7. The method of claim 1, wherein at least a part of the generated guidance information is matched to at least one of the information on the medical appointment or electronic medical record of the patient and the context information of the patient, among information stored in a database, and is extracted from the database.

8. The method of claim 1, wherein the providing step comprises the steps of:
determining whether the patient enters the hospital, with reference to the information on the location of the device of the patient;
providing the generated guidance information to the device of the patient, when it is determined that the patient enters the hospital.

9. A non-transitory computer-readable recording medium having stored thereon a computer program for executing the method of claim 1.

10. A system for providing a medical service, comprising:
a location recognition unit configured to determine whether a patient carrying a patient device enters a hospital, with reference to information on a location of the patient device;
an information management unit configured to acquire information on a medical appointment or an electronic medical record (EMR) of the patient, when it is determined that the patient enters the hospital; and
a medical resource management unit configured to establish a medical resource allocation policy personalized for the patient, with reference to the acquired information on the medical appointment or electronic medical record, wherein the information management unit is further configured to generate guidance information to be provided to the patient from a guidance information database, with reference to the information on the medical appointment or electronic medical record of the patient and context information of the patient, to determine a time or place in which the generated guidance information is to be provided to the patient, and to provide the generated guidance information to the device of the patient, when the determined time has arrived or the patient is located at the determined place, and
wherein the context information includes information on at least one of a medical history of the patient at the hospital or other hospitals, a disease of the patient, a medicine taken by the patient, a lifestyle of the patient, and a biometric signal acquired from the patient.

11. The system of claim 10, wherein the location of the patient device is determined on the basis of information acquired from the patient device or at least one wireless communication module that is installed in the hospital and performs short-range wireless communication with the patient device.

12. The system of claim 10, wherein the location of the patient device is determined on the basis of information acquired from an inertial measurement unit (IMU) provided in the patient device.

13. The system of claim 10, wherein the medical resources include at least one of medical attendants, medicines, medical equipment, beds, and medical care hours.

14. The system of claim 10, wherein the medical resource management unit is configured to establish the medical resource allocation policy personalized for the patient, with further reference to information on at least one of a current location of the patient and a past location history of the patient.

15. The system of claim 10, wherein the medical resource management unit is further configured to distribute the established medical resource allocation policy to at least one hospital device or hospital server that manages the medical resources of the hospital.

16. The system of claim 10, wherein at least a part of the generated guidance information is matched to at least one of the information on the medical appointment or electronic medical record of the patient and the context information of the patient, among information stored in a database, and is extracted from the database.

17. The system of claim 10, wherein the information management unit is configured to determine whether the patient enters the hospital, with reference to the information on the location of the device of the patient, and to provide the generated guidance information to the device of the patient, when it is determined that the patient enters the hospital.
